(19) United States
(12) Patent Application Publication
(10) Pub. No.: US 2013/0165944 A1
(43) Pub. Date: Jun. 27, 2013

(54) APPARATUS, SYSTEMS, AND METHODS FOR REMOVING OBSTRUCTIONS IN THE URINARY TRACT

(71) Applicant: The Board of Trustees of the Leland Stanford Jr. University, Palo Alto, CA (US)

(72) Inventors: David Gal, San Francisco, CA (US); Raymond Bonneau, San Francisco, CA (US); Mary K. Garrett, Redwood City, CA (US); Dan E. Azagury, Geneve (CH)

(73) Assignee: The Board of Trustees of the Leland Stanford Jr. University, Palo Alto, CA (US)

(21) Appl. No.: 13/716,001

(22) Filed: Dec. 14, 2012

Related U.S. Application Data
(60) Provisional application No. 61/576,308, filed on Dec. 15, 2011.
(60) Provisional application No. 61/604,330, filed on Feb. 28, 2012.
(60) Provisional application No. 61/604,400, filed on Feb. 28, 2012.
(60) Provisional application No. 61/647,354, filed on May 15, 2012.
(60) Provisional application No. 61/662,662, filed on Jun. 21, 2012.
(60) Provisional application No. 61/676,607, filed on Jul. 27, 2012.

(51) Int. Cl. A61B 17/22

(52) U.S. Cl. CPC A61B 17/22
USPC 606/127

(57) ABSTRACT

Systems and methods are provided for removing an obstruction from a ureter using a catheter including a distal end sized for introduction into a ureter, an infusion lumen, and an aspiration lumen. An infusion tip is provided on the distal end that includes infusion ports communicating with the infusion lumen, and one or more aspiration ports communicating with the aspiration lumen. One or more sources of fluid and/or vacuum are connectable to the tubular member such that fluid is delivered into the infusion lumen and suction is delivered into the aspiration lumen. A controller is coupled to the source(s) to control delivery of fluid to generate a radially outward pressure against a wall of a ureter adjacent the infusion tip and to control suction through the aspiration port(s) to apply a suction force against a kidney stone located adjacent the infusion tip.

### RELATED APPLICATION DATA

This application claims benefit of co-pending provisional application Ser. Nos. 61/576,308, filed Dec. 15, 2011, 61/604,330, filed Feb. 28, 2012, 61/604,400, filed Feb. 28, 2012, 61/647,354, filed ______, 61/662,662, filed Jun. 21, 2012, and 61/676,607, filed Jul. 27, 2012. The entire disclosures of these applications are expressly incorporated by reference herein.

### FIELD OF THE INVENTION

The present invention generally relates to apparatus, systems, and methods for removing obstructions from within a patient's body, and more particularly to apparatus, systems, and methods for removing obstructions, such as calculi, from a patient's urinary tract or other body lumens.

### BACKGROUND

Ureteral calculi (kidney stones) are a significant burden on society and the health care system. Kidney stones are formed when the saturation of various minerals in urine exceeds a metastable limit and a precipitate is formed. The majority of stones are comprised of calcium and oxalate, though uric acid, struvite, cysteine, and other stone compositions are also commonly found.

Stones are typically formed in the renal pelvis or calyces and can stay there for years. When a stone becomes dislodged, it makes its way down the upper urinary tract towards the bladder though the ureter. The ureter is a naturally collapsed tube with an inner diameter on the order of about one to three millimeters (1-3 mm). As a result, stones often get stuck en route to the bladder in the ureter. Even extremely small stones may become stuck in the ureter. One reason for this is that mechanical rubbing of the sharp stone on the ureter's mucosal lining may cause an inflammatory response and edema, which inhibits the stone's ability to pass. This obstruction impedes the passage of urine from the kidney to the bladder, which results in increased internal pressure of the kidney. This pressure rise causes the volume of the kidney to increase, which causes the nerve fibers in the kidney to stretch, which in turn results in the excruciating pain well known to accompany stones. Clinically this pain is known as “renal colic” and typically presents as unexpected bursts of two to eighteen (2-18) hours until the internal pressure of the kidney is reduced. So long as the stone remains in the urinary tract, patients are at risk for renal colic. Pain relief may be substantially instantaneous after stone passage or removal.

Various methods have been proposed for removing such stones. For example, Extracorporeal Shockwave Lithotripsy (ESWL) is a procedure in which shockwaves are transmitted through the body in the direction of the stone in an attempt to fragment it into smaller pieces. ESWL requires a specialized bed on which the patient lies while their body is bombarded with shockwaves. Ureteroscopy (URS) is a procedure in which a urologist inserts an endoscope up the urethra, into the bladder, and finally up the ureter to the site of the stone. Using a laser, the urologist fragments the stone into smaller pieces and retracts the fragments. Percutaneous Nephrectomy Lithotripsy (PCNL) is a surgical procedure in which a tube is inserted through the back into the kidney. Stones are removed through the tube using lasers, graspers, and aspiration.

Existing procedures require anesthesia including both general and conscious sedation, specialized facilities, and expensive equipment. For example, URS requires anesthesia due to requisite constant irrigation of the kidney and extreme mechanical manipulation in the urinary tract. During ESWL, shockwaves are applied directly to the kidney region, which induces significant pain and, therefore, requires a minimum of conscious sedation. In order to surgically enter the kidney, PCNL requires general anesthesia. Introducing an anesthesia mortality risk for a non-fatal condition is undesirable.

Therefore, a simple method and apparatus for removing urinary tract obstructions without directly impacting the kidney are desirable.

### SUMMARY

The present invention is directed to apparatus, systems, and methods for removing obstructions from within a patient's body, and more particularly to apparatus, systems, and methods for removing obstructions, such as calculi, from a patient's urinary tract or other body lumens.

In accordance with an exemplary embodiment, a method is provided for facilitating movement of a kidney stone through a ureter of a patient that includes introducing fluid into a portion of the ureter between the kidney stone and the patient's urinary bladder to dilate the ureter, e.g., to reduce frictional forces on the stone, and applying suction to at least one of the introduced fluid or the kidney stone such that the kidney stone moves towards the bladder. For example, the suction may be used to apply a force directly to the stone, e.g., to facilitate pulling the stone towards the bladder, and/or the suction may be used to maintain a desired pressure and/or volume of fluid within the ureter adjacent the stone.

In accordance with another embodiment, a method is provided for facilitating movement of a kidney stone through a ureter of a patient that includes introducing a distal end of a tubular member from the bladder into the ureter until the distal end is disposed adjacent the kidney stone; and delivering fluid through the distal end into the ureter to dilate the ureter, e.g., to reduce frictional forces exerted by the walls of the ureter on the kidney stone. The reduction in friction may allow the kidney stone to move towards the bladder with a reduced “removal” force, e.g., simply under gravity. Optionally, an additional removal force may be applied, such as those caused by peristaltic activity of the ureter, suction, flushing with water or other fluid, and/or gravity, which may facilitate and/or cause movement of the kidney stone towards the bladder.

In accordance with still another embodiment, a method is provided for facilitating movement of a kidney stone through a ureter of a patient that includes introducing a distal end of a tubular member from the patient's bladder into the ureter; advancing the distal end past the kidney stone; and introducing fluid through the distal end into the ureter to dilate the ureter and/or apply an antegrade force to the kidney stone, e.g., such that the antegrade force exceeds the ureter-stone frictional force.

In accordance with yet another embodiment, a method is provided for facilitating movement of a kidney stone through a ureter of a patient that includes introducing a distal end of a tubular member into the patient's bladder; substantially isolating the ureter from the bladder; introducing fluid from the tubular member into the ureter; and regulating at least one of volume, pressure, and flow rate of the fluid introduced into the ureter to dilate the ureter such that the ureter-stone frictional forces are reduced to facilitate movement of the kidney stone towards the bladder. For example, the tubular member may be introduced only into the bladder, into the ureterovesical junction, or into the ureter until disposed adjacent the kidney stone.

In accordance with still another embodiment, a system is provided for facilitating movement of a kidney stone through a ureter that includes a tubular member comprising a proximal end, a distal end sized for introduction into a ureter, and an infusion lumen, an aspiration lumen, and a longitudinal axis extending between the proximal and distal ends; and an infusion tip on the distal end of the tubular member comprising a plurality of infusion ports communicating with the infusion lumen, and one or more aspiration ports communicating with the aspiration lumen.

One or more sources of fluid and vacuum are connectable to the proximal end of the tubular member such that fluid is delivered from the one or more sources into the infusion lumen and suction is delivered from the one or more sources into the aspiration lumen; and a controller is coupled to the one or more sources, e.g., to control infusion of fluid through the infusion ports to generate a radially outward pressure against a wall of a ureter adjacent to the infusion tip and to control suction through the one or more aspiration ports to apply a suction force proximally along the longitudinal axis against a kidney stone located adjacent the infusion tip.

In an exemplary embodiment, the infusion ports are located around an outer surface of the infusion tip, and the one or more aspiration ports are located distal to the infusion ports on the infusion tip. For example, the infusion tip may have a substantially flat distal surface, and the one or more aspiration ports include an aspiration port in the distal surface. In exemplary embodiments, the outer surface of the infusion tip may have a substantially uniform cylindrical shape, or an expanding cylindrical shape such that the distal surface has a larger diameter than the distal end of the tubular member. In addition or alternatively, the infusion ports may define an angle relative to the longitudinal axis such that fluid injected through the infusion ports is directed radially outwardly and distally to apply a radial and distal force to a ureter wall surrounding a kidney stone.

In accordance with yet another embodiment, a system is provided for facilitating movement of an obstruction through a ureter communicating with a bladder of a body that includes a tubular member including a proximal end, a distal end sized for introduction into at least one of a bladder and a ureter, and an infusion lumen extending between the proximal end and one or more infusion ports in the distal end. A source of fluid is connectable to the proximal end for delivering fluid through the infusion lumen out the one or more infusion ports into a ureter. A sensor may be provided for sensing a parameter including at least one of pressure, flow rate, and volume of the fluid delivered into the ureter; and a controller may be coupled to the source of fluid and the sensor to adjust at least one of the pressure, flow rate, and volume of the fluid delivered into the ureter based on the sensed parameter to modify dilation of the ureter and cause an obstruction in the ureter to move through the ureter towards the bladder.

In accordance with another embodiment, a system is provided for facilitating movement of a kidney stone through a ureter communicating with a bladder of a body that includes a tubular member including a proximal end, a distal end sized for introduction into a ureter, an infusion lumen extending from the proximal end to a plurality of infusion ports in an infusion tip on the distal end, and an aspiration lumen extending from the proximal end to one or more aspiration ports offset proximally from the infusion tip; and one or more sources of fluid and vacuum connectable to the proximal end of the tubular member such that fluid is delivered from the one or more sources into the infusion lumen and suction is delivered from the one or more sources into the aspiration lumen.

A controller may be coupled to the one or more sources for applying suction to the aspiration lumen to draw a region of the ureter wall against an outer surface of the tubular member adjacent the one or more aspiration ports to at least partially isolate a section of the ureter distally beyond the region of the ureter wall, the controller coupled to the one or more sources for delivering fluid through the infusion ports via the infusion lumen into the isolated section of the ureter.

In accordance with another embodiment, an apparatus is provided for facilitating movement of a kidney stone through a ureter of a patient that includes an elongate body having a proximal end, a distal end sized for introduction into a ureter; at least one fluid introduction lumen in the elongate body for introducing fluid into the ureter; at least one fluid introduction aperture at or near the distal end of the elongate body and in fluid communication with the at least one fluid introduction lumen; at least one suction lumen in the elongate body for applying suction to at least one of the fluid or the kidney stone; at least one suction aperture at or near the distal end of the elongate body and in fluid communication with the at least one suction lumen; and at least one stone detection member at or near the distal end of the elongate body for helping locate the stone within the ureter.

In accordance with still another embodiment, a system is provided for facilitating movement of a kidney stone through a ureter of a patient that includes an elongate body having a proximal end, a distal end sized for introduction into a ureter; at least one fluid introduction lumen in the elongate body for introducing fluid into the ureter; at least one fluid introduction aperture at or near the distal end of the elongate body and in fluid communication with the at least one fluid introduction lumen; at least one suction lumen in the elongate body for applying suction to at least one of the fluid or the kidney stone; at least one suction aperture at or near the distal end of the elongate body and in fluid communication with the at least one suction lumen; at least one stone detection member at for helping locate the stone within the ureter; and a source of fluid and/or controller removably couplable with the elongate body at or near its proximal end for regulating a flow rate of fluid introduced into the ureter and a flow rate of fluid suctioned out of the ureter.

These and other aspects of the present invention are apparent in the following detailed description and claims, particularly when considered in conjunction with the accompanying drawings in which like parts bear like reference numerals.

### BRIEF DESCRIPTION OF THE DRAWINGS

The drawings illustrate exemplary embodiments, in which:

FIG. 1A is a side view of an exemplary embodiment of an apparatus for removing stones from a body lumen of a patient.

FIG. 1B is a side view of a distal portion of the apparatus of FIG. 1A.

FIGS. 2A-2C show alternative embodiments of tips that may be provided on the apparatus of FIGS. 1A and 1B.

FIGS. 3A-3H are schematic views of flow patterns that may be generated using various tips of the apparatus of FIGS. 1A and 1B, such as the tips shown in FIGS. 2A-2C.

FIGS. 4A-4C are cross-sectional views of a patient's ureter showing an exemplary method for removing a stone using the apparatus of FIGS. 1A and 1B.

FIG. 5 is a cross-sectional view of a patient's ureter showing another method for removing a stone using the apparatus of FIGS. 1A and 1B.

FIGS. 6A-6C are cross-sectional views of a patient's bladder and ureter showing yet another method for removing a stone using the apparatus of FIGS. 1A and 1B along with a capture device.

FIG. 7 is a cross-sectional view of a patient's ureter showing another method for removing a stone using a column of fluid.

FIG. 8 is a cross-sectional view of a patient's body, showing yet another method for removing a stone using a porous balloon catheter.

### DETAILED DESCRIPTION OF THE EXEMPLARY EMBODIMENTS

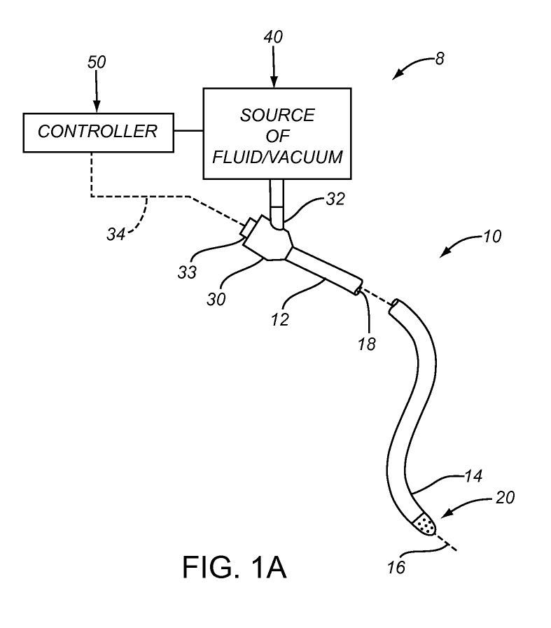

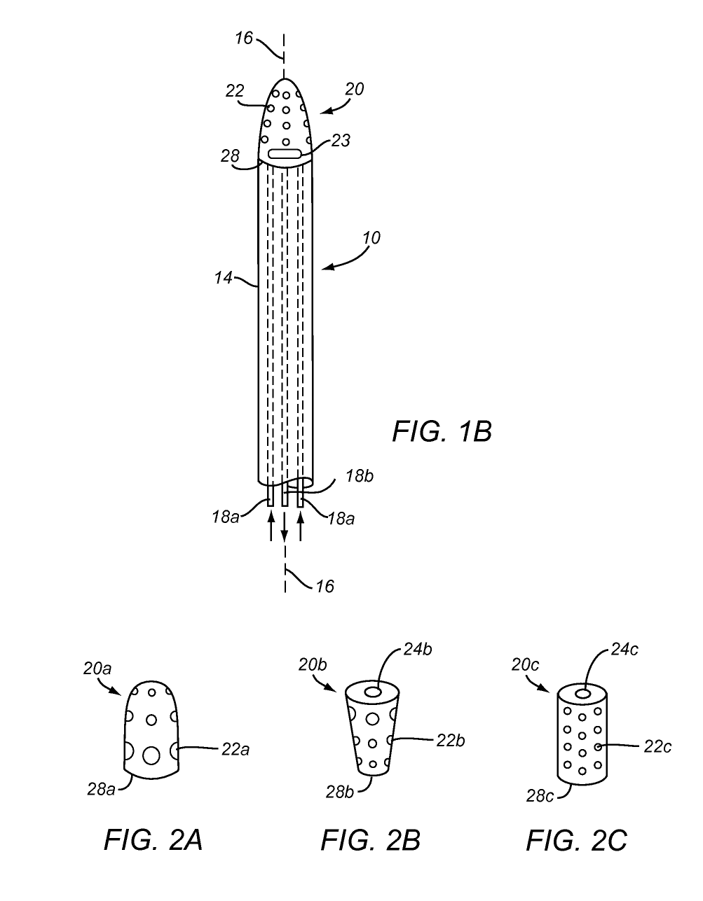

Turning to the drawings, FIGS. 1A and 1B show an exemplary embodiment of an apparatus 8 for removing obstructions from within a patient's body, such as calculi from a patient's urinary tract or other body lumens (not shown). Although the apparatus, systems, and methods herein are generally described with reference to removing kidney stones within a ureter, it will be appreciated that they may be used in other body lumens of a human or animal patient in addition to the urinary tract, to remove gallstones and/or blood clots.

Generally, the apparatus 8 includes a catheter or other elongate tubular member 10, one or more sources of fluid and/or vacuum 40, e.g., one or more pumps and/or syringes (not shown), and a controller or console 50 for operating the source(s) of fluid and/or vacuum 40. Optionally, the apparatus 10 may be provided as a kit or system including one or more additional components, such as a guidewire, cystoscope, guide catheter, and/or other delivery device, a capture device, and the like (not shown), as described elsewhere herein.

As shown in FIG. 1A, the catheter 10 generally includes a proximal end 12, a distal end 14 sized for insertion into a body lumen, a central longitudinal axis 16 extending therebetween, and one or more lumens 18 extending between the proximal and distal ends 12, 14. In the exemplary embodiment shown in FIG. 1B, the catheter 10 may include one or more infusion lumens 18a (e.g., two shown) coupled to a source of fluid (not shown), and one or more aspiration lumens 18b (e.g., one shown) coupled to a source of vacuum (not shown). Optionally, a guidewire or accessory lumen (not shown) may be provided that extends between the proximal and distal ends 12, 14, e.g., if the catheter 10 is introduced over a guidewire or other rail (not shown) into a patient's body.

The catheter 10 may have a substantially uniform construction along its length, or alternatively, the construction may be varied. For example, in one embodiment, the proximal end 12 may be substantially rigid or semi-rigid, e.g., providing sufficient column strength to allow the catheter 10 to be pushed from the proximal end 12, while the distal end 14 may be substantially flexible or semi-rigid. Thus, the distal end 14 of the catheter 10 may be advanced or otherwise manipulated within a patient's body from the proximal end 12 without substantial risk of buckling and/or kinking Optionally, if desired, one or more steering elements (not shown) may be provided that extend between the proximal and distal ends 12, 14 for bending or otherwise steering the distal end 14, e.g., to facilitate introducing the distal end 14 into a body lumen, such as the ureter from the bladder, as described further elsewhere herein.

The catheter 10 may be sized such that the distal end 14 may be introduced into a body lumen, such as the urethra and/ureter, e.g., directly or through a cystoscope, guide catheter, or other delivery device. In exemplary embodiments, the catheter 10 may have an outer diameter between about one and four millimeters (1-4 mm), and a length between about fifty and one hundred twenty centimeters (50-120 cm). Optionally, the distal end 14 and/or a distal portion of the catheter 10 may be tapered and/or have a smaller diameter than the proximal end or proximal portion, e.g., to facilitate insertion into and/or advancement within the patient's body.

In addition, the catheter 10 includes an infusion tip 20 on the distal end 14 including one or more ports 22 communicating with the lumen(s) 18 of the catheter 10, e.g., for delivering fluid into a body lumen and/or evacuating fluid from a body lumen, as described further below. The tip 20 may be integrally formed from the same material as the distal end 14 of the catheter 10. For example, the distal end 14 of the catheter 10 may be molded to include a plurality of ports in the wall thereof, or a plurality of ports may be drilled, laser cut, or otherwise formed in the wall.

Alternatively, the tip 20 may be formed from separate material, e.g., metal, plastic, or composite material, including a proximal end 28 that is attached to the distal end 14, e.g., by bonding with adhesive, heat flowing or fusing, interference fit, one or more connectors (not shown), and the like. Optionally, the tip 20 may be formed from or include radiopaque material, echogenic material e.g., to facilitate identifying the tip 20 using fluoroscopy, ultrasound, or other external imaging.

The infusion tip 20 may have a desired shape and/or port pattern, such as those shown in FIGS. 2A-2C. For example, the tip 20a shown in FIG. 2A may provide a rounded, tapered, and/or otherwise substantially atraumatic distal tip for the catheter 10, e.g., to facilitate advancement within a body lumen and/or around a stone, as described elsewhere herein. Alternatively, the tip 20b shown in FIG. 2B may have an increasing tapered shape, which may enhance a desired pattern of jet streams out of the ports 22b, enhance contact with a stone adjacent the tip 20b, and/or at least partially isolate a region distally beyond the tip 20b, e.g., to enhance a suction force applied to a stone (not shown) adjacent the tip 20b. In a further alternative shown in FIG. 2C, the tip 20c may have a substantially uniform, e.g., cylindrical, shape along its length.

In addition or alternatively, the tip 20 may include a predetermined array of ports 22 to provide a desired pattern of jet streams of fluid delivered through the catheter 10. For example, the array of ports 22 may include ports having different sizes, e.g., diameters, along the length of the tip 20, to provide desired variations in velocity, force, and/or flow rate of fluid from ports 22 along the length of the tip 20. In the embodiment shown in FIG. 1B, in addition to a plurality of substantially circular ports 22, the tip 20 also includes one or more elongated circumferential ports or slots 23. In addition or alternatively, the ports 22 may have different angular orientations relative to the longitudinal axis 16 of the catheter 10, e.g., substantially perpendicular to, transversely and distally (i.e., distally away from the distal end 14 of the catheter 10), and/or transversely and proximally (i.e., proximally back towards the distal end 14 of the catheter 10 to which the tip 20 is attached) relative to the longitudinal axis 16.

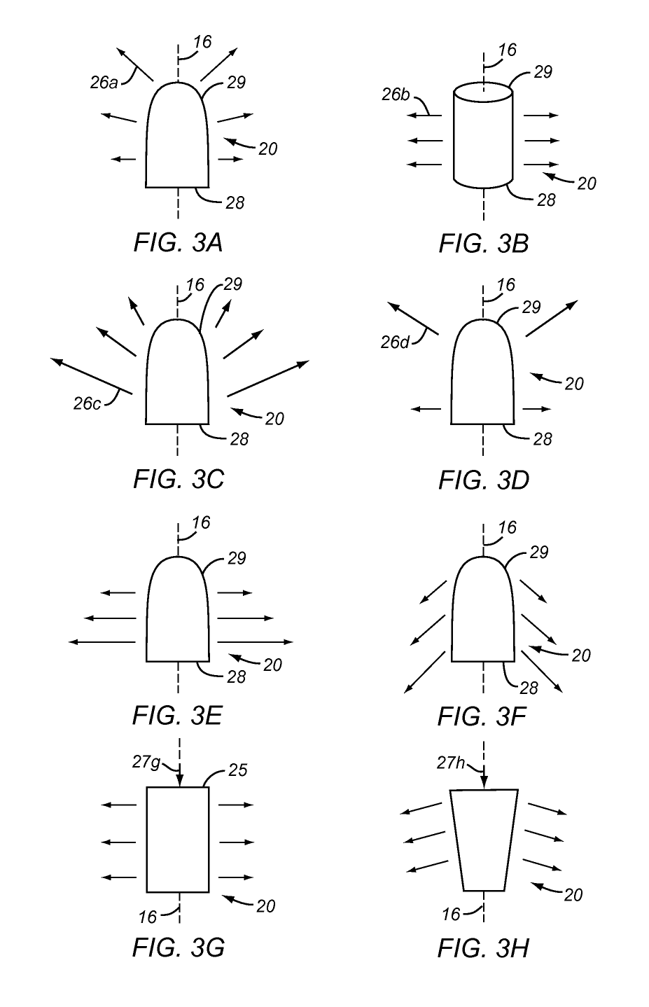

FIGS. 3A-3H show exemplary velocity, force, and/or flow patterns of jet streams that may be achieved using different tips 20 of the catheter 10, as described further elsewhere herein. The tips 20 may be configured to provide a plurality of streams of fluid having desired velocities, flow rates, and/or directions of fluid flow (as represented by arrows 26), e.g., to apply desired forces against the wall of the ureter, stone, and the like. For example, FIGS. 3A and 3D show exemplary flow patterns of jet streams 26a, 26d that may be achieved with an array of ports (not shown for simplicity) having greater velocities and/or flow rates out the ports closer to the distal end than the proximal end 28 of the tip 20. The ports may also be configured such that the primary direction of fluid flow of the jet streams 26a, 26d from the tip 20 (and resulting forces) is substantially perpendicular to or transversely and distally relative to the longitudinal axis 16. FIGS. 3B and 3E show exemplary flow patterns of jet streams 26b, 26e that have a primary flow direction substantially perpendicular to the longitudinal axis 16, with the velocities (and resulting forces) of the flow pattern 26b in FIG. 3B being substantially uniform along the length of the tip 20.

The velocities (and resulting forces) of the flow pattern 26e in FIG. 3E may taper from the proximal end 28 to the distal end 29 of the tip 20. For example, the size and/or spacing of the ports 22 may be modified along the tip 20 from the proximal end 28 towards the distal end 29, e.g., by providing smaller cross-sectional area ports 22 and/or spacing the ports 22 further apart along the length of the tip 20. FIG. 3C shows a flow pattern of jet streams 26c that has greater velocities and/or flow rates out ports adjacent the proximal end 28 than the distal end 29, and has a primary flow direction (and resulting force direction) that is oriented transversely and distally towards and beyond the distal end 29. For example, in the embodiment shown in FIG. 1B, one or more circumferential ports or slots 23 may be provided adjacent the proximal end of the tip 20, e.g., to provide greater flow rates, velocities, and/or forces adjacent the proximal end than those provided by the smaller circular ports 22 towards the distal end of the tip 20.

In exemplary embodiments, the ports may be configured to generate jet streams 26c that define an angle relative to the longitudinal axis 16 between about ninety degrees (substantially perpendicular) and about thirty degrees (distally) (90-30°). In contrast, FIG. 3F shows a flow pattern of jet streams 26f that has a primary flow direction that is oriented transversely and proximally back towards and beyond the proximal end 28, e.g., also between about ninety degrees (substantially perpendicular) and about thirty degrees (distally) (90-30°).

FIGS. 3G and 3H show exemplary flow patterns of jet streams for tips 20g, 20h that include both supply jets 26g, 26h and aspiration or suction jets 27g, 27h. For example, FIG. 3G shows a flow pattern that includes a substantially uniform velocity and/or force pattern of supply jet streams 26g that are directed radially outward and substantially perpendicular to the longitudinal axis 16, and a localized aspiration jet stream 27g that is directed substantially axially and/or proximally. FIG. 3H shows a pattern of supply jet streams 26h that may be more isolated from an aspiration jet stream 27h, e.g., due to the increased tapered shape and/or larger distal surface 25h of the tip 20h, which may enhance application of a suction force against a stone (not shown) disposed immediately adjacent the distal surface 25h. In addition, the larger surface area of the distal surface 25h and corresponding suction port (not shown) may enhance the suction force, engagement, and/or otherwise pulling on the stone. Thus, the tip 20 of FIG. 3H (and others herein) may provide substantially simultaneous localized infusion forces and suction forces, which may be at least partially isolated from one another by the shape of the tip 20 and/or the location of the infusion and aspiration ports. It will be appreciated that one or more aspiration jet streams may be provided in any of the embodiments shown herein, e.g., by providing one or more aspiration lumens and/or ports on the tip 20, to apply desired suction forces against a stone or other obstruction, as described elsewhere herein.

Optionally, returning to FIGS. 1A and 1B, the distal end 14 may also include one or more additional features, e.g., one or more markers, sensors (for example, one or more pressure sensors, flow sensors, electrical sensors, and/or impedance sensors), an imaging device, and the like (not shown), on the tip 20 and/or on the distal end 14 of the catheter 10 adjacent the tip 20, as described elsewhere herein. For example, one or more radiopaque bands or other markers (not shown) may be provided on the distal end 14 and/or the tip 20, e.g., to facilitate identifying the distal end 14 using fluoroscopy or other external imaging.

In addition or alternatively, one or more sensors (not shown) may be provided on the tip 20 or distal end 14, which may be coupled to the controller 50 to measure desired parameters, such as pressure, velocity, flow rate, and the like, as described elsewhere herein. In another exemplary embodiment, one or more sensors on the tip 20 and/or distal end 14 may be configured to measure one or more mechanical parameters of the ureter or other region around the distal end 14, e.g., strain and/or elasticity of the wall of the ureter, vibrational signals, and the like. For example, a pair of coils, bands, or other electrodes (not shown) may be provided on the tip 20 or distal end 14, which may be coupled to the controller 50 to measure electrical impedance or other parameters, as described further elsewhere herein. In yet another embodiment, an imaging device, e.g., a CMOS, fiberoptic, or other device (not shown) may be provided on the tip 20 or distal end 14 to allow imaging distally beyond the tip 20, e.g., to provide direct visualization within a body lumen.

In addition or alternatively, the distal end 14 may include one or more additional ports (not shown) offset proximally from the tip 20, which may communicate with the infusion and/or aspiration lumens 18 of the catheter 10, as desired. Such additional ports may facilitate generating additional desired infusion and/or suction forces relative to the distal end 14.

Optionally, the infusion lumen or aspiration lumen (or another lumen, not shown, in the tip 20) may be used for other functions in addition to or instead of infusion and/or aspiration, e.g., to provide a channel to facilitate visualization, stone detection, or other stone sensing mechanism. For example, the lumen may communicate with one or more sensors in the proximal end 12 of the catheter 10 and/or in the controller 50 coupled to the proximal end 12, as described further elsewhere herein. Alternatively, a wire or other transducer may be introduced into the lumen to a desired location, e.g., into the distal end 14, and used to measure pressure and/or other parameters. In addition or alternatively, the aspiration lumen may be used to advance the catheter 10 over a guidewire or other rail (not shown). In yet another alternative, a fiberoptic or other imaging device (not) shown may be introduced into the aspiration lumen (or other axial lumen), e.g., for imaging distally beyond the tip 20.

As shown in FIG. 1A, the proximal end 12 may include a handle or hub 30 including one or more ports 32, e.g., communicating with respective lumens 18 of the catheter 10, one or electrical connectors 33, e.g., for coupling the catheter 10 to the controller 50, and/or other features. For example, if the catheter 10 includes a pair of infusion lumens 18a, as shown in FIG. 1B, a pair of infusion ports (not shown) may be provided on the handle 30, e.g., communicating with the respective infusion lumens 18a. Similarly, if the catheter 10 includes an aspiration lumen 18b, as shown in FIG. 1B, an aspiration port (not shown) may be provided on the handle 30 communicating with the aspiration lumen 18b. Thus, in the embodiment of FIG. 1B, three ports may be provided on the handle (not shown) to allow source(s) of fluid and/or vacuum to be coupled to the respective ports or two ports (also not shown) may be provided if the catheter 10 includes a single infusion lumen and a single aspiration lumen. Alternatively, a single port may be provided that includes separate passages communicating with the respective lumens 18, and a corresponding connector (not shown) may be provided for coupling to the port to allow communication between the appropriate lumens 18 and the source(s) of fluid and/or vacuum 40. Optionally, the port(s) 32 may include one or more valves, e.g., a hemostatic valve, Luer fitting, and the like (not shown), which may provide a substantially fluid-tight seal and/or otherwise facilitate connecting the source(s) of fluid and/or vacuum 40 to the catheter 10.

In addition, if the catheter 10 includes one or more sensors and/or electrodes, e.g., within the handle 30 and/or on the distal end 14, one or more electrical connectors, and the like (one exemplary connector 33 shown in FIG. 1A) may be provided on the handle or hub 30 for coupling the sensor(s) and/or electrode(s) to another device, such as the controller 50 shown in FIG. 1A (as shown schematically by 34). Alternatively, the hub 30 may be connected directly to the controller 50 (not shown), which may make all necessary connections for operating the system 8. Optionally, the handle 30 may include one or more actuators, such as sliders, buttons, switches, and the like (not shown), e.g., for activating and/or manipulating components (also not shown) on the distal end 14 or otherwise operating the apparatus 10. For example, a switch (not shown) may be provided on the handle 30 for turning the source(s) of fluid and/or vacuum 40 and/or the controller 50 on or off, adjusting their operating parameters, and the like. In addition or alternatively, if the distal end 14 of the catheter 10 is steerable, an actuator (not shown) may be provided that is coupled to a steering element for deflecting the distal end 14 of the catheter 10 in a desired manner.

With continued reference to FIGS. 1A and 1B, the source(s) of fluid and/or vacuum 40 may include one or more devices for delivering fluid into the infusion lumen(s) 18a of the catheter 10 and/or aspirating fluid through the aspiration lumen(s) 18b. In an exemplary embodiment, a pump device may be provided that includes a source of fluid, e.g., a syringe or other container, a fluid line, and the like (not shown), an impeller or motor, a plunger, and the like (also not shown), which may deliver one or more desired fluids in a desired manner. For example, the pump device may be configured to provide one or more of a substantially continuous stream of fluid (or suction), an intermittent, e.g., pulsed, stream of fluid, a bolus of fluid, a volume of fluid limited by predetermined pressure thresholds, flow rates, and the like, as described further elsewhere herein.

The pump device may include one or more fluids, e.g., within a housing (not shown), therein for delivery via the catheter 10. For example, the fluid may simply be saline or other water-based solution, or may include viscous or semi-viscous materials. Optionally, the fluid may include one more pharmaceutical agents, such as a biocompatible lubricant, an analgesic, e.g., a mixture of lidocaine and KY), a ureter relaxing agent, contrast, and the like. Optionally, the fluid may include particles to enhance imaging, dilation, and/or treatment, e.g., magnetic particles, as described elsewhere herein. For example, ferromagnetic particles may be suspended within the fluid, e.g., having diameters or other outer dimensions on the order of several micrometers.

In addition, in some embodiments, the source 40 may also include a pump device, syringe, and the like (not shown) configured to provide a vacuum to aspirate or otherwise remove fluid via the catheter 10, simultaneously or alternatively with the delivery of fluid. For example, a single pump device may be alternated between infusion and aspiration, or a plurality of separate pump devices may be configured to provide a substantially steady state of simultaneous or alternating fluid delivery and aspiration to maintain desired parameters within the patient's body lumen, such as a predetermined fluid flow rate, pressure, volume, and the like. The source of vacuum 40 may also be used to provide suction force to “attach” onto the stone and/or provide a proximal or antegrade force to remove the stone from the ureter (“antegrade” meaning in the direction from the kidney towards the bladder, as during normal flow), as described elsewhere herein. For example, as described further elsewhere herein, the source of vacuum may be operated to cause fluid upstream of the stone to flow around the stone into the catheter 10, e.g., flushing the fluid around the stone to apply a proximal force that pushes, pulls, or otherwise moves the stone along the ureter.

The controller 50 may be coupled to the source(s) of fluid and/or vacuum 40, e.g., to control operation of the pump device(s). For example, the pump device(s) (or the controller 50 itself) may include sensors to measure desired parameters of fluid delivered into and/or aspirated from the catheter 10, e.g., fluid pressure, flow rate, and the like, and the controller 50 may control the pump device(s) to maintain these parameters at desired values or ranges. Alternatively, the pump device(s), e.g., one or more syringes, may be operated manually by the user rather than being operated manually by an electronic controller. Such manual operation may be performed tactilely, based on feedback from one or more sensors, or otherwise, similar to the methods performed by the controller 50 and described elsewhere herein.

Optionally, the catheter 10 may include one or more sensors, e.g., a pressure sensor, flow rate sensor, and the like (not shown) in the handle 50 communicating with a lumen extending between the proximal and distal ends 12,14, on the tip 20 and/or distal end 14, or elsewhere as desired, which may be coupled to the controller 50 such that the controller 50 operates the pump device(s) based on parameters measured within the patient's body, as described further elsewhere herein. For example, a sensor may be provided in the handle 30, the pump device(s) 40, and/or the controller 50 that communicates with the infusion lumen 18a to measure flow rate, pressure, and/or other parameters of fluid delivered into the ureter. Similarly, a sensor may also be provided that communicates with the aspiration lumen 18b to measure flow rate, pressure, and/or other parameters of fluid aspirated from the ureter. Alternatively, separate lumens may be used for measuring such parameters. In another alternative, a wire or other transducer (not shown) may be introduced into the catheter 10, e.g., through one of the ports 32 into an associated lumen 18 to measure one or more desired parameters.

In an exemplary embodiment, the controller 50 may monitor pressure and/or flow rate to ensure that a desired pressure within the ureter adjacent the stone is not exceeded, e.g., to reduce the risk of excessive discomfort or injury to the patient. In another embodiment, the controller 50 may monitor pressure to detect a predetermined change in pressure or other threshold, e.g., a pressure increase that may occur when the tip 20 of the catheter 10 is advanced into a ureter to a position immediately adjacent the stone to be removed. For example, the controller 50 may direct the pump to deliver fluid in a desired steady state, e.g., substantially continuously or intermittently, and monitor the pressure and/or other parameters during the delivery, while the catheter 10 is being advanced within the ureter. When the fluid pressure increases (or a rate of increase in fluid pressure increases), e.g., due to the tip 20 being located adjacent the stone, the controller 50 may determine that the tip 20 is in a desired position and should no longer be advanced.

In addition or alternatively, the controller 50 may use other algorithms to locate the tip 20, e.g., using a Fourier transform to detect changing frequencies in a time signal, for example, to detect changes in magnitude and/or phases of pressure, or using a Laplace transform to detect non-time domain changes. The controller 50 may provide an output, e.g., on a display or other indicator (not shown) on the controller 50 or on the handle 30 of the catheter 10, to inform the user of such changes, e.g., to notify the user to discontinue advancement, which may facilitate the user manipulating the catheter 10 and/or otherwise facilitating removal of the stone.

In another alternative, the controller 50 may monitor one or more parameters of the source(s) of fluid and/or vacuum 40 during fluid infusion. For example, a sensor for monitoring the power consumption of an electrical pump, a flow sensor, e.g., a hall effect sensor for measuring RPMs of the pump motor, or other sensor may be provided on the pump, e.g., for measuring one or more of current, voltage, speed, and/or other parameters of the pump, and coupled to the controller 50, which may include an algorithm that uses the pump parameters as a surrogate signal to indicate pressures and/or forces being delivered by the fluid into the ureter.

In still another alternative, the controller 50 may be coupled to one or more sensors on the distal end 14 of the catheter 10 to obtain data regarding the ureter or other region adjacent the distal end 14. For example, one or more impedance or other sensors on the distal end 14 may provide data that the controller 50 may correlate to wall strain and/or elasticity of the wall of the ureter. For example, electrical signals, stimulus signals including a DC or AC component, may be applied between a pair of spaced-apart sensors contacting the wall of the ureter, and the controller 50 may monitor the response signals to determine strain and/or elasticity data of the wall, which may be correlated to pressure, force, or other parameters created by fluid delivery into the ureter. In addition or alternatively, the controller 50 may correlate impedance signals from the sensors to determine when the distal end 14 is immediately adjacent a kidney stone, e.g., by identifying a change in impedance that may occur within the ureter between locations where only fluid and/or tissue are present and the location where the stone is present.

In yet another alternative, the controller 50 may be coupled to one or more electrodes (not shown) on the distal end 14 of the catheter 10 to apply electrical energy to the wall of the ureter. For example, the controller 50 may direct electrical pulses or other signals to the wall via the electrode(s), e.g., direct current signals, sinusoidal signals, and/or other periodic or aperiodic signals, which may cause electrical exhaustion, relaxation, and/or other responses by the ureter. Such responses may reduce wall tensions and/or other forces and/or induce localized contraction and/or expansion of the ureter, which may reduce frictional forces on the stone and/or allow the stone to move through the ureter.

In another embodiment, the controller 50 may control the source(s) of fluid and/or vacuum 40 to induce peristalsis of the ureter. For example, the controller 50 may cause fluid flow to be toggled dynamically, e.g., to induce a peristaltic wave, which may enhance stone removal, as described further elsewhere herein. Peristalsis is a natural smooth muscular contractile wave the ureter uses to pass boluses of urine from the kidney to the bladder. This contractile wave may help naturally pass stones by pushing the stone towards the bladder. The controller 50 may pulse and/or otherwise deliver fluid such that the jet streams injected into the ureter apply pressure, e.g., in a pulsatile manner. Such pulsing may be of sufficient pressure on the ureter wall to “trick the ureter” into thinking a bolus of urine is going through it and thus induce peristalsis. Such pulsing may press against the wall of the ureter, e.g., similar to a surgeon pinching the ureter, which may induce one or more, e.g., a series of, peristaltic waves within the ureter to push the stone towards the bladder. Thus, the controller 50 may enhance or modify natural amplitudes and/or frequencies of peristalsis, which may facilitate moving a stone through the ureter.

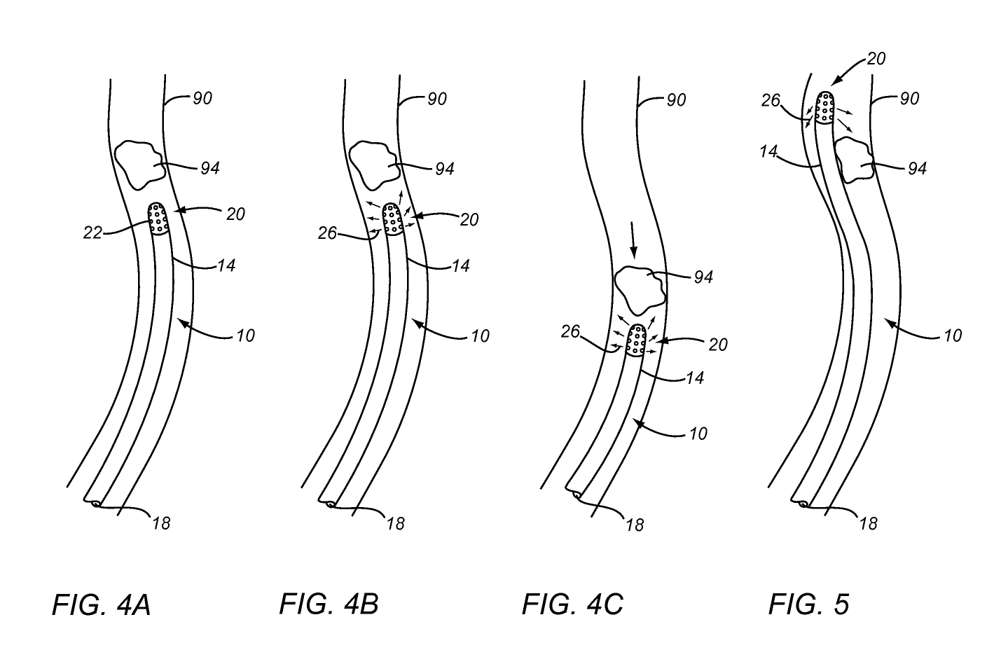

Turning to FIGS. 4A-4C, an exemplary method is shown for facilitating removal of a kidney stone or other obstruction 94 within a patient's ureter or other body lumen 90, e.g., using the apparatus 8 shown in FIG. 1A. Initially, the distal end 14 of the catheter 10 may be introduced into the patient's body, e.g., via the urethra into the bladder and through the ureterovesical junction (not shown) into the ureter 90.

Optionally, the distal end 14 may be introduced through another device (not shown), e.g., previously introduced into the bladder. For example, a distal end of a cystoscope or other device (not shown) may be introduced into the bladder using conventional methods, and oriented towards the ureterovesical junction, e.g., using direct visualization from a camera or other imaging device on the distal end of the cystoscope. For example, if the cystoscope is steerable and sized to be received within the ureter, the distal end may be introduced into the bladder, and manipulated to insert the distal end into the ureterovesical junction. The distal end 14 of the catheter 10 may then simply be advanced through an accessory lumen of the cystoscope into the ureter 90. Alternatively, the distal end of the cystoscope may remain within the bladder, e.g., with the accessory lumen aligned with the ureterovesical junction, and the distal end 14 of the catheter 10 may be advanced through the cystoscope and into the ureter 90. In a further alternative, if the distal end 14 of the catheter 10 is steerable, the distal end 14 may be introduced into the bladder, e.g., through a cystoscope or other device, and then manipulated to align and insert the distal end 14 into the ureter 90.

In yet another alternative, a guidewire or other rail (not shown) may be introduced into the ureter, e.g., through a channel of a cystoscope or other delivery device introduced into the bladder and/or ureter 90, and the distal end 14 of the catheter 10 may be advanced over the guidewire. For example, the guidewire may be back loaded through the aspiration lumen, an infusion lumen, or a dedicated lumen (not shown), and then the distal end 14 may be advanced over the guidewire to a desired position within the ureter 90. The guidewire may be removed once the distal end 14 is located within the ureter 90 or may remain within the catheter 10 until before infusion/aspiration or at any other desired time during the procedure.

During introduction and subsequent fluid delivery and/or treatment, the distal end 14 of the catheter 10 may be monitored using external imaging, e.g., fluoroscopy, ultrasound, and the like. For example, contrast may be injected through one or more of the lumens of the catheter 10 into the ureter 90 during advancement, and fluoroscopy may be used to monitor the location of the tip 20, e.g., using one or more markers on the tip 20 or distal end 14, until the tip 20 is disposed adjacent the obstruction 94. Alternatively, ultrasound may be used, which may facilitate monitoring fluid flow relative to the tip 20 and/or obstruction 94. Such imaging methods may be used at any desired times during the treatment.

As shown in FIG. 4A, the distal end 14 of the catheter 10 may be advanced into the ureter 90 until the tip 20 is disposed adjacent the obstruction 94, e.g., until the tip 20 is disposed directly below the obstruction 94. As shown in FIG. 4B, fluid may then be delivered from the tip 20, e.g., as represented by arrows 26, according to desired treatment parameters to dilate the ureter 90 and/or otherwise facilitate movement of the obstruction 90 through the ureter 90 towards the bladder. For example, jets of fluid (from the source 40 shown in FIG. 1A) may be injected from the tip 20 outwardly towards the wall of the ureter 90 to facilitate dilation of the wall. The jets may be injected substantially continuously or pulsed, e.g., to produce or apply desired pressures, flow rates, velocities, forces, and/or volumes within the ureter 90 to dilate the wall, e.g., as controlled by the controller 50. The resulting dilation and/or fluid injection may reduce friction between the obstruction 94 and the wall, and/or dislodge the obstruction 94, thereby facilitating removal. The resulting dilation may not stretch or otherwise expand the wall of the ureter, but may simply open the ureter sufficiently to reduce friction and allow the obstruction 94 to travel naturally along the ureter. Alternatively, the wall may be stretched to increase a diameter or other cross-section of the ureter, if desired, e.g., without causing any damage to the wall, e.g., such that friction is reduced or to a diameter larger than the obstruction 94 such that the obstruction simply passes, e.g., under gravity, through the ureter 90. In addition, although not shown in FIG. 4-C, suction may also be applied, and the resulting dilation may increase the diameter or other cross section of the ureter 90 sufficiently such that the combination of suction force and gravity overcomes the friction force.

The fluid delivered into the ureter 90 may be free to flow around the catheter 10 back towards the bladder. In addition or alternatively, along with injection of fluid, vacuum may be applied to control the pressure and/or volume of fluid present within the ureter 90. For example, as described above, one or more of the ports 22 in the tip 20 may communicate with a source of vacuum to aspirate fluid from the ureter 90 in a desired flow pattern and/or to maintain a desired pressure and/or volume within the ureter 90. Alternatively, as shown in FIGS. 2B and 2C, the tip 20b, 20c may include an aspiration port 24b, 24c oriented distally, e.g., on the distal end of the tip 20b, 20c such that localized vacuum is directed towards the obstruction 94 while force and/or pressure from the delivered fluid is localized towards the wall of the ureter 90. Similar to the infusion flow, the aspiration flow may be substantially continuous or intermittent, e.g., pulsed or otherwise varied in flow rate, pressure, volume, and the like, to apply desired suction forces on the obstruction 94. In this alternative, the localized vacuum may apply a force to pull the obstruction 94 towards the bladder, e.g. possibly pulling the obstruction 94 against the distal end of the tip 20, while the fluid dilation force may free the obstruction 94 from the wall and/or otherwise reduce friction such that the vacuum may pull the obstruction 94 along the ureter 90.

Optionally, the catheter 10 may include one or more additional infusion and/or aspiration ports (not shown) offset from the tip 20, e.g., at one or more locations along the distal end 14 adjacent the tip 20. For example, additional fluid may be injected adjacent the tip 20 to enhance dilation of the wall of ureter 90 between the obstruction 94 and the bladder. In addition or alternatively, fluid within the ureter 90 may be aspirated from around the distal end 14 via additional aspiration ports on the distal end 14, e.g., to maintain desired fluid volume and/or pressure within the ureter 90.

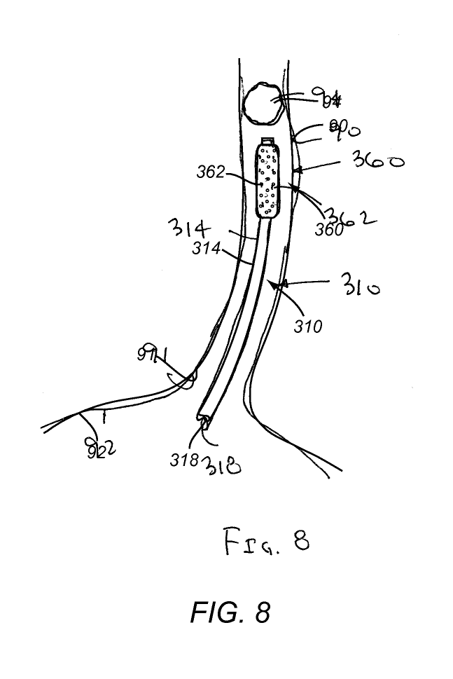

In a further alternative, shown in FIG. 8, a catheter 310 may be provided that includes a porous balloon 360 on the distal end 314, e.g., instead of the tip 20, or in addition to the tip 20, if desired. The balloon 360 may include a plurality of pores 362 therein for delivering fluid, e.g., via lumen 318, similar to other embodiments herein. The material of the balloon 360 and/or the pore size of the pores 362 may be such that the pores remain substantially closed below a threshold pressure. For example, initial fluid delivery into the balloon 360 via the lumen 318 may cause the balloon 360 to expand with the pores 362 substantially sealed. Once the threshold pressure is exceeded, the pores 362 may open to allow fluid within the balloon 360 to be ejected into the ureter 90 to provide localized dilation. The threshold pressure may be set using one or more of a constraining mesh around or attached to the balloon 360, by an additional internal or external balloon (not shown), by a controller (not shown) used to control fluid flow into the balloon 360, by engineering the shape, wall thickness, pore size, and/or other mechanical properties of the balloon 360, and the like. Thus, similar to the tip 20, the balloon 360 may inject jet streams radially outwardly towards the wall of the ureter or otherwise into the ureter, similar to other embodiments herein. Optionally, the catheter 310 may also include an aspiration lumen, e.g., including a suction port (not shown) on the distal end 314 beyond the balloon 360, which may used to apply a suction force on the obstruction 94 and/or fluid, similar to other embodiments herein.

Optionally, if the catheter 10 includes multiple infusion lumens communicating with respective ports, the controller 50 may alternate or otherwise vary delivery of fluid through the respective infusion lumens to dynamically change the forces applied to the ureter by the catheter 10. For example, by pulsing or otherwise varying jet streams applied at different locations of the ureter 90 adjacent the distal end 14, different regions of the ureter 90 may be dilated greater than others to enhance releasing the obstruction 90, to induce desired peristaltic waves, and the like.

In another alternative, one or more aspiration ports may be provided at a predetermined location on the distal end 14 offset from the tip 20 such that application of the vacuum causes the wall of the ureter 90 to be pulled against the wall of the catheter 10 at the location, e.g., to at least partially seal the ureter 90, e.g., to isolate a region of the ureter between the location and the obstruction 94. This alternative may facilitate controlling fluid volume and/or pressure within the ureter 90 adjacent the obstruction 94, e.g., to facilitate localized dilation of the wall of the ureter 90.

As shown in FIG. 4C, the catheter 10 may be withdrawn while delivering and/or aspirating fluid, thereby dilating the local wall of the ureter 90 adjacent the tip 20 and the obstruction 94, thereby allowing the obstruction 94 to travel freely and/or be pulled down the ureter 90 towards and into the bladder. Once within the bladder, the obstruction 94 may be captured and/or otherwise removed using known procedures.

Optionally, as described elsewhere herein, the distal end 14 of the catheter 10 may include one or more sensors, e.g., a pressure sensor (not shown) on or adjacent to the tip 20. For example, a pressure sensor may be used to obtain localized pressure within the ureter 90 adjacent the obstruction 94, with the controller 50 operating the source of fluid 40 to maintain a desired pressure within the ureter 90 to enhance dilation without damaging the wall of the ureter 90. For example, the controller 50 may obtain pressure readings from the sensor to maintain a desired pressure range within the ureter, e.g., below a maximum pressure, to provide a safe and effective pressure level, e.g., under one hundred ninety millimeters of Mercury (190 mmHg), to reduce patient pain but above some lower pressure threshold desired for sufficient dilation. Alternatively, a pressure sensor may be placed proximally, for instance, in the handle 30, in the pump, or at the controller 50, and pressure may be monitored in this location, e.g., knowing the translation between measured pressures at the sensor, and the resultant pressures at the tip 20 and/or wall of the ureter. In an exemplary embodiment, it may be desirable to generate pressures at the wall of the ureter between about forty to seventy millimeters of Mercury (40-70 mm HG) to provide sufficient dilation without causing excessive pain or injury to the ureter. If sensors at locations other than the distal end 14 are used to determine the pressure at the ureter, the controller 50 may use algorithms to correlate pressures at the sensor, which may be substantially higher than the pressure within the ureter, to approximate the pressure and resulting forces being applied to dilate the ureter 90.

Turning to FIG. 5, an alternative method is shown for removing an obstruction 94 from the ureter 90. Similar to the previous embodiments, the distal end 14 of the catheter 10 may be introduced into the ureter 90 adjacent the obstruction 94. In this alternative, the distal 14 may be directed past the obstruction 94, as shown, and then fluid may be delivered, as represented by 26, to dilate the wall of the ureter 90 and/or otherwise facilitate removal of the obstruction 94. FIG. 5 shows an exemplary flow pattern, which may be similar to that shown in FIG. 3F, where the primary orientation of flow is transversely and proximally to enhance dilation locally around the obstruction 94. As the obstruction 94 is released and begins to travel down the ureter 90, the distal end 14 of the catheter 10 may be withdrawn to maintain substantially localized dilation around the obstruction 94, e.g., with the tip 20 remaining beyond the obstruction 90, until the obstruction 90 passes into the bladder. In addition, in this embodiment, the orientation of the jet streams from the tip 20 may be directed proximally towards the obstruction 94 to apply forces against the obstruction 94 to facilitate pushing or otherwise moving the obstruction 94 through the ureter 90 once released from the wall of the ureter 90.

In another alternative, if desired, particles or other material (not shown) may be delivered with the fluid to enhance treatment during any of the procedures described herein. For example, ferromagnetic particles may be included in the fluid that may be injected into the ureter 90 adjacent the obstruction 94. In this alternative, an external magnetic field may then be applied, e.g., using a device placed on the patient's skin or otherwise adjacent the ureter 90 outside the patient's body, to cause the particles to move radially outwardly and/or otherwise to enhance localized dilation of the ureter 90. In a further alternative, an internal magnetic field may be applied, e.g., using a generator on the distal end 14 of the catheter 10 and/or using another device (not shown) introduced into the ureter 90, which may repel the particles outwardly to dilate the wall of the ureter 90.

In a further alternative, particles may be injected or otherwise delivered into the patient's body upstream of the obstruction 94. For example, fluid carrying the particles may be injected intravenously into the patient's body, and sufficient time may pass to allow the particles to enter the ureter above the obstruction 94, e.g., until particles are disposed around the obstruction 94. A magnetic field may then be applied to dilate the ureter 90 and/or otherwise facilitate the obstruction 94 moving through the ureter 90 towards the bladder. In addition or alternatively, the magnetic field may be used to apply a distal force on the particles, which may apply a corresponding force against the obstruction 94 to facilitate advancing the obstruction 94 through the ureter 90.

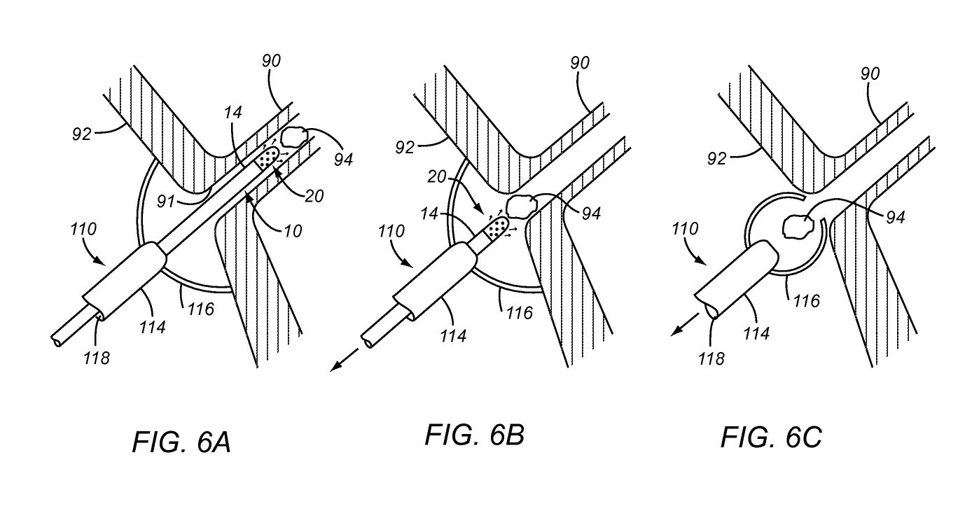

Turning to FIGS. 6A-6C, another method for removing a kidney stone or other obstruction is shown, e.g., using the apparatus 10 of FIG. 1A (or any other apparatus described herein). In this embodiment, a capture device 110 may also be provided, e.g., as part of a system including the apparatus 10. For example, the capture device 110 and catheter 10 may be provided in a telescoping or other configuration in which the distal end 14 of the catheter 10 may be deployed from and/or withdrawn into or through the capture device 110. Alternatively, the catheter 10 may be introduced outside, e.g., adjacent the capture device 110 such that the catheter 10 and capture device 110 are movable independently of one another.

Generally, the capture device 110 includes a proximal end (not shown), a distal end 114 sized for introduction into a body lumen, e.g., the patient's urethra and bladder, and one or more lumens 118 extending therebetween, e.g., similar to the catheter 10. For example, the capture device 110 may include an accessory lumen 118 through which the distal end 14 of the catheter 10 may be received and/or deployed. In addition, the capture device 110 includes a capture mechanism 116 on the distal end 114, e.g., for capturing an obstruction released by the apparatus 10. In an exemplary embodiment, the capture mechanism 116 may be an inverted umbrella, basket, or other expandable structure, which may be movable between an open configuration, such as that shown in FIGS. 6A and 6B, and a closed configuration, such as that shown in FIG. 6C. Alternatively, the capture mechanism 116 may be provided directly on the distal end 14 of the fluid delivery catheter 10, e.g., offset proximally from the tip 20.

For example, the capture mechanism 116 may be actuatable from the proximal end of the capture device 110, e.g., such that a user may selectively open and close the capture mechanism 116. Alternatively, the capture mechanism 116 may be biased to one of the open and closed configurations, but may be directed to the other configuration during use. For example, the capture mechanism 116 may be biased to the open configuration, yet may be selectively closed, e.g., using one or more filaments or other features (not shown) on the open end of the capture mechanism 116 to pull the open end closed.

During use, the capture device 110 may be introduced through the patient's urethra into the bladder 92 and manipulated to place the capture mechanism 116 in the open configuration adjacent the ureterovesical junction 91. For example, the distal end 114 may be introduced through the urethra into the bladder 92 with the capture mechanism 116 in a collapsed configuration, e.g., the closed configuration or in a third configuration smaller than the closed configuration. Similar to the methods above, the capture device 110 may be introduced through an accessory lumen of a cystoscope or other delivery device (not shown), similar to other embodiments herein. Once within the bladder 92, the capture mechanism 116 may be opened and placed against the wall of the bladder 92 adjacent the ureterovesical junction 91, as shown in FIG. 6A.

The distal end 14 of the apparatus 10 may then be introduced, e.g., through a lumen 118 of the capture device 110, through the bladder 92 into the ureter 90. The distal end 14 may be advanced within the ureter 90 until disposed adjacent an obstruction 94, e.g., immediately below the obstruction 94 as shown in FIG. 6A, and fluid may be delivered into the ureter 90 from the tip 20, e.g., similar to the method shown in FIGS. 4A-4C. Alternatively, the tip 20 may be introduced into the ureter 90 beyond the obstruction 94, similar to the method shown in FIG. 5.

As fluid is injected and/or aspirated from the ureter 90 to locally dilate the wall of the ureter 90 and release the obstruction 94, the catheter 10 may be withdrawn as shown in FIG. 6B, e.g., causing or allowing the obstruction 94 to travel along the ureter 90 towards and into the bladder 92. Once the obstruction 94 enters the bladder 92 beyond the ureterovesical junction 91, the capture mechanism 116 may be used to capture the obstruction 94 for removal. For example, with the distal end 14 of the catheter 10 removed, the capture mechanism 116 may be directed to the closed configuration, as shown in FIG. 6C. In an alternative embodiment, the capture mechanism 116 may be provided on the distal end 14 of the catheter 10 (not shown), e.g., such that a single device is introduced into the bladder. In this alternative, the capture mechanism 116 may be offset from the tip 20 sufficiently such that the tip 20 may be introduced a desired distance into the ureter 90, yet close enough such that the capture mechanism 116 captures the obstruction 94 once directed into the bladder 92. The capture device 110 and obstruction 94 may then be removed from the bladder 92. It will be appreciated that a capture device may be used in cooperation with any of the embodiments herein.

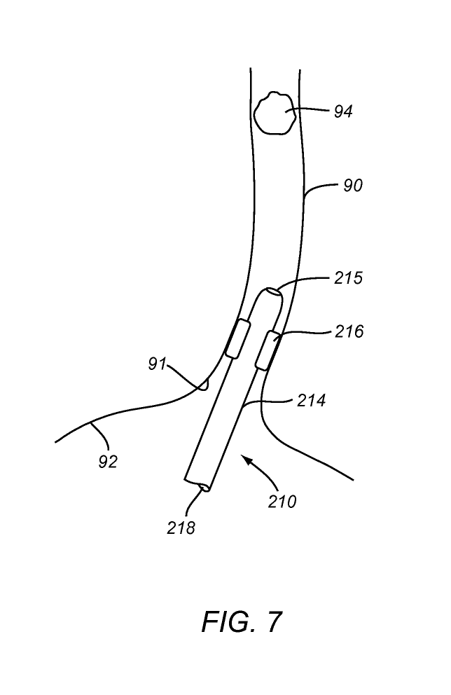

Turning to FIG. 7, another method is shown for facilitating removal of a kidney stone or other obstruction 94 from within a ureter 90 or other body lumen within a patient's body using an apparatus 208. Generally, the apparatus 208 includes a catheter or other tubular member 210 including a proximal end (not shown), a distal end 214 sized for introduction into a body lumen, e.g., the patient's urethra and bladder, and one or more lumens 218 extending therebetween, e.g., similar to the catheter 10 shown in FIGS. 1A and 1B. In addition, the apparatus 208 may include a pump device or other source of fluid and/or vacuum, and a controller (not shown), similar to that shown in FIG. 1A.

Unlike the catheter 10, the catheter 210 includes an occlusion member 216 on the distal end 214, e.g. for substantially isolating the ureter 90, e.g., between the obstruction 94 and the bladder 92. In an exemplary embodiment, the occlusion member 216 may be a balloon or other expandable member, e.g., formed from elastic and/or compliant material, that is expandable from a contracted condition to an enlarged or occluding condition. In the embodiment shown in FIG. 7, the occlusion member 216 may be expandable to a diameter larger than the ureter 90 and/or the ureterovesical junction 91, e.g., to a maximum diameter between about five and eight millimeters (5-8 mm).

The catheter 210 may include an outlet 215 in the distal end 215, e.g., distally beyond the occlusion member 216, that communicates with the lumen 218. The lumen 218 may communicate with the source of fluid and/or vacuum, e.g., via a port on the proximal end (not shown) of the catheter 210, similar to other embodiments.

During use, with the occlusion member 216 in its contracted condition, the distal end 214 of the catheter 210 may be introduced via the urethra into the bladder 92, e.g., using a cystoscope or other delivery device, similar to other embodiments herein. Optionally, external imaging, e.g., fluoroscopy or ultrasound, may be used during introduction and/or use of the catheter 210, similar to other embodiments herein. Once within the bladder 92, the distal end 214 may be manipulated to insert the occlusion member 216 into the ureterovesical junction 91, and then the occlusion member 216 may be expanded to the occluding condition, e.g., to substantially seal the ureter 90, as shown in FIG. 7. Alternatively, the occlusion member 216 may be expanded within the bladder 92 and then the expanded occlusion member 216 may be pressed against the ureterovesical junction 91 to isolate the ureter 90. In a further alternative, the distal end 214 may be advanced into the ureter until the distal end 214 is positioned adjacent the obstruction 94, whereupon the occlusion member 216 may be expanded to isolate the region between the occlusion member 216 and the obstruction 94. In still a further alternative, the occlusion member 216 may be expanded within the urethra or within the bladder to at least partially isolate the ureter 90.

In yet another alternative, an expandable bowl or dome-shaped member (not shown) may be provided on the distal end 214 of the catheter 210 instead of the occlusion member 216, which may be introduced into the bladder 92 in a contracted condition and released or otherwise expanded to an expanded condition within the within the bladder 92. The edges of the dome-shaped member may then be pressed against the wall of the bladder 92 surrounding the ureterovesical junction 91 to substantially isolate the ureter 90 from the bladder 92.

In still another alternative, the distal end 214 of the catheter 210 may include one or more side ports, e.g., a plurality of ports disposed radially around the distal end 214 (not shown), instead of the occlusion member 216. The side ports may communicate with one or more aspiration lumens extending to the proximal end of the catheter 210, e.g., similar to other embodiments herein. Once the distal end 214 is introduced into a desired location within the ureter 90, suction may be applied via the side ports to draw the wall of the ureter against the wall of the catheter 210, thereby substantially sealing and/or isolating the ureter 90 distally beyond the side ports from the bladder.

With the ureter 90 at least partially isolated and/or sealed, fluid may be delivered via the lumen 218 into the ureter 90 towards the obstruction 94. The controller may control fluid delivery to provide desired parameters within the ureter 90 adjacent the obstruction 94, e.g., a desired volume, pressure, and/or flow rate, similar to other embodiments herein. For example, sufficient fluid may be delivered such that a column of fluid fills the ureter 90, e.g., between the ureterovesical junction 91 and the obstruction 94, as shown in FIG. 7. Similar to other embodiments herein, the fluid may cause localized dilation of the wall of the ureter 90 adjacent the obstruction 94, thereby releasing the obstruction 94 and/or reducing friction between the obstruction 94 and the wall of the ureter 90. The fluid may be controlled such that the obstruction 94 can then move through the ureter 90 towards and into the bladder 92.

For example, if the obstruction 94 substantially occludes the ureter, a column of water having a desired volume and/or pressure may be created within the ureter 90 until the obstruction 94 is released from the wall. If additional fluid is needed to maintain the desired water column volume and/or pressure, the controller may operate the source of fluid to deliver additional fluid as needed. Optionally, if less fluid is needed, fluid delivery may be discontinued, pulsed, and/or reversed, e.g., to aspirate some of the fluid from within the ureter 90. Alternatively, a separate aspiration lumen (not shown) may be provided in the catheter 210 for aspirating fluid from within the ureter 90, similar to other embodiments herein.

Optionally, similar to other embodiments herein, a pressure sensor may be provided on the distal end 214 of the catheter 210, e.g., beyond the occlusion member 216, or within a handle or hub (not shown), e.g., communicating with a lumen of the catheter 210, to allow the controller to obtain pressure readings, similar to other embodiments herein. Alternatively, the pressure sensor may also be located within the source(s) of fluid and/or vacuum or at the controller (not shown), e.g., with the distal pressure determined through translation of a known pressure ratio. In addition or alternatively, external imaging may be used to monitor fluid flow, dilation, and/or other parameters during fluid delivery and/or removal of the obstruction 94.

In addition or alternatively, the controller may control fluid delivery dynamically, e.g., to induce peristalsis and/or mimic a peristaltic wave, if desired to facilitate movement of the obstruction 94. In a further alternative, if desired, magnetic particles may be provided in the fluid and a magnetic field may be applied to enhance dilation, similar to other embodiments herein.

In another alternative embodiment, fluid may be delivered beyond the obstruction 94, e.g., to at least partially fill the ureter 90, the renal pelvis, and/or calyces (not shown). Pressure, volume, and/or other parameters of the fluid may be controlled to maintain the parameter(s) below predetermined maximum threshold(s), e.g., to prevent excessive and/or painful pressure to the patient. When the desired maximum threshold(s) are achieved, the water column may be reduced or otherwise modified, e.g., to allow the column of water and obstruction to pass from the ureter 90 into the bladder 92 to facilitate removal of the obstruction 94. Optionally, the passage of water may be timed with natural or induced peristalsis, to enhance movement of the obstruction 94 through the ureter 90. If desired, multiple columns of water may be created and/or drained within the ureter 90 to enhance movement and/or removal of the obstruction 94.

Optionally, in any of the embodiments described herein, additional treatment may be used in conjunction to facilitate movement and/or removal of an obstruction within the ureter. For example, internal or external sources of energy or other treatments may be applied to the patient, such as sonication, cavitation air bubbles, injected gas bubbles, ureteral wall vibration, focused ultrasound, acoustic waves, shockwave lithotripsy, and the like. For example, external energy sources, such as a focused ultrasound system could be placed adjacent the patient's body and used to direct energy towards the obstruction. Optionally, bubbles may be included within the fluid delivered into the ureter, which may cavitate or otherwise react to the energy to enhance movement and/or destruction of the obstruction. For example, fluidic (air, gas, etc) bubbles may be used to provide forces to dislodge and/or facilitate movement of the stone through the ureter.

It will be appreciated that elements or components shown with any embodiment herein are exemplary for the specific embodiment and may be used on or in combination with other embodiments disclosed herein.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope of the appended claims.

1. A method for facilitating movement of an obstruction through a ureter communicating with a bladder of a body, the method comprising:
introducing fluid into a portion of the ureter between the obstruction and the bladder to dilate a wall of the ureter to reduce a frictional force exerted by the ureter wall on the obstruction; and
applying suction to at least one of the introduced fluid or the obstruction such that the obstruction moves towards the bladder.

2. The method of claim 1, wherein the introduced fluid exerts an amount of force on the ureter wall such that removal forces applied to the obstruction are greater than the frictional force exerted by the ureter wall on the obstruction, wherein the removal forces comprise at least one of suction, gravity, pulling force applied by a basket or other pulling member on a removal device, or hydrodynamic force generated by the introduced fluid.

3. The method of claim 2, wherein the removal forces include peristaltic force.

4. The method of claim 3, wherein the fluid is introduced into the ureter in such a way as to induce peristaltic contractions of the ureter to produce the peristaltic force.

5. The method of claim 1, wherein the suction is applied to the fluid in such a way as to generate a turbulent or circular flow pattern in the fluid around the obstruction to dislodge the obstruction from contact with the wall of the ureter.

6. The method of claim 1, further comprising:
capturing the obstruction with a capturing device; and
removing the obstruction from the ureter by withdrawing the capturing device through the ureter.

7. The method of claim 1, further comprising capturing the obstruction in the bladder.

8. The method of claim 1, further comprising detecting a location of the obstruction relative to a distal tip of a fluid delivery device used to introduce the fluid.

9. The method of claim 1, wherein fluid is injected radially outwardly from a fluid delivery device towards the ureter wall, and wherein suction is applied from a distal tip of the fluid delivery device to draw the obstruction towards the tip.

10. The method of claim 1, wherein introducing the fluid comprises:
introducing a an elongate member into the ureter; and
passing fluid through at least one fluid introduction aperture in the elongate member in pressurized streams to generate a dilating force against the ureter wall.

11. The method of claim 10, wherein passing fluid comprises injecting streams of fluid from a plurality of apertures in the elongate member such that the streams are directed at an angle relative to a longitudinal axis of the elongate member.

12-22. (canceled)

23. The method of claim 1, wherein the fluid is introduced in a pulsatile flow or other flow pattern configured to induce peristalsis of the ureter.

24. A method for facilitating movement of an obstruction through a ureter communicating with a bladder of a body, the method comprising:
introducing fluid into a portion of the ureter between the obstruction and the bladder to dilate a wall of the ureter to reduce a frictional force exerted by the ureter wall on the obstruction;
measuring a parameter including at least one of diameter of the ureter or pressure, flow rate, or volume of the fluid introduced into the ureter; and
adjusting at least one of the pressure, flow rate, or volume of the fluid introduced into the ureter based on the measured parameter to modify dilation of the ureter and to cause the obstruction to move through the ureter towards the bladder.

25. The method of claim 24, wherein the introducing, measuring and adjusting steps are performed by a system of coupled device components.

26. The method of claim 24, wherein the measuring and adjusting steps are performed at least partially manually by a user and are not automatically controlled by a system.

27. The method of claim 24, further comprising applying suction to the obstruction to enhance movement of the obstruction towards the bladder.

28. The method of claim 24, further comprising occluding the ureter at a location between the obstruction and the bladder before or during introducing the fluid.

29. A system for facilitating movement of a kidney stone through a ureter communicating with a bladder of a body, the system comprising:
a tubular member comprising a proximal end, a distal end sized for introduction into a ureter, an infusion lumen, an aspiration lumen, and a longitudinal axis extending between the proximal and distal ends;
one or more infusion ports at or near the distal end of the tubular member and in fluid communication with the infusion lumen;
one or more aspiration ports at or near the distal end of the tubular member and in fluid communication with the aspiration lumen;
one or more sources of fluid and suction connectable to the tubular member at or near its proximal end such that fluid is delivered from the one or more sources into the infusion lumen and suction is delivered from the one or more sources into the aspiration lumen; and
at least one controller coupled to the one or more sources to control infusion of fluid through the infusion ports to generate a radially outward pressure against a wall of a ureter near the infusion tip and to control suction through the one or more aspiration ports to apply a suction force proximally along the longitudinal axis against a kidney stone located near the infusion tip.

30. The system of claim 29, wherein the controller comprises a manual controller, operable by a user, for manually adjusting amounts of infused fluid and suction applied via the tubular member, and wherein the user may make adjustments via the controller based upon user-observed feedback.

31. The system of claim 29, wherein the infusion ports are located on a side wall of the tubular member, and wherein the one or more aspiration ports are located distal to the infusion ports, closer to the distal end.

32. The system of claim 29, wherein the infusion ports are configured to infuse fluid outwardly from the tubular member at an angle relative to the longitudinal axis.

33. The system of claim 29, wherein the infusion tip has a tapered distal portion to facilitate advancing the infusion tip through a ureter without snagging on or causing trauma to the wall of the ureter.

34. The system of claim 29, wherein the controller is configured to operate the one or more sources to introduce fluid at a predetermined flow rate, pressure, or volume to exert an amount of force on the ureter wall such that removal forces applied to the kidney stone are greater than frictional forces exerted by the ureter wall on the kidney stone, wherein the removal forces comprise at least one of suction, gravity, pulling force applied by a basket or other pulling member on a removal device, peristaltic force generated by peristalsis of the ureter, or hydrodynamic force generated by the introduced fluid.

35. The system of claim 29, wherein the controller is configured to operate the one or more sources to apply radial infusion forces and a suction force locally to different regions within the ureter.

36. The system of claim 29, further comprising a sensor for measuring a parameter of fluid delivered by the one or more sources, and wherein the controller is coupled to the sensor for measuring the parameter, the controller configured to operate the one or more sources to modify fluid infused through the infusion ports.

37. The system of claim 36, wherein the sensor comprises a pressure sensor communicating with the infusion lumen.

38. The system of claim 29, further comprising an electrical impedance sensor on the tubular member at or near the distal end, the controller coupled to the impedance sensor for measuring impedance signals and determining one or more properties within the ureter based on the measured impedance signals, the controller configured to operate the one or more sources to modify at least one of the fluid infused through the infusion ports or the suction force based on the one or more determined properties.

39-42. (canceled)

43. The system of claim 29, wherein the controller is configured to operate the one or more sources to infuse the fluid through the infusion ports to induce peristalsis of the ureter.

44. The system of claim 29, further comprising a capture device on the tubular member at or near the distal end configured to capture a kidney stone removed from the ureter by the tubular member.

45. An apparatus for facilitating movement of a kidney stone through a ureter of a patient, the apparatus comprising:
an elongate body having a proximal end, a distal end and a diameter sized to pass through at least a portion of the ureter;
at least one fluid introduction lumen in the body for introducing fluid into the ureter;
at least one fluid introduction aperture at or near the distal end of the body and in fluid communication with the at least one fluid introduction lumen;
at least one suction lumen in the body for applying suction to at least one of the fluid or the kidney stone;
at least one suction aperture at or near the distal end of the body and in fluid communication with the at least one suction lumen; and
at least one sensor for sensing one or more parameters at or near the distal end of the elongate body.

46-51. (canceled)
